(19) United States
(12) Patent Application Publication
(10) Pub. No.: US 2012/0003314 A1
(43) Pub. Date: Jan. 5, 2012

(54) DELAYED-RELEASE ORAL PHARMACEUTICAL COMPOSITION FOR TREATMENT OF COLONIC DISORDERS

(75) Inventors: Vinay H. SHUKLA, Columbus, OH (US); Eric M. SPILLER, Columbus, OH (US)

(73) Assignee: ROXANE LABORATORIES, INC., Columbus, OH (US)

(21) Appl. No.: 13/233,523

(22) Filed: Sep. 15, 2011

Related U.S. Application Data
(63) Continuation of application No. 11/965,151, filed on Dec. 27, 2007, now abandoned.

(51) Int. Cl. A61K 9/36; A61K 31/606; B05D 5/00; A61P 1/00; A61P 1/04; B05D 1/36; A61K 9/00; A61K 9/28

(52) U.S. Cl. 424/480; 424/400; 514/166; 514/163; 514/161; 424/474; 427/2.21

(57) ABSTRACT

A timed or delayed release oral composition delivery system for the treatment of colonic disorders and diseases is provided. According to one aspect, a delayed release oral pharmaceutical composition includes an active core comprising a therapeutically effective amount of 5-amino salicylic acid (i.e., mesalamine); a primary coating composition disposed around the active core, wherein the primary coating composition includes an enteric polymer; and a secondary coating composition disposed around the primary coating composition, wherein the secondary coating composition includes a ratio mixture of ethyl cellulose and hydroxypropyl methylcellulose.

### TECHNICAL FIELD

The present invention is directed in general to an orally-administrable pharmaceutical composition that comprises 5-amino-salicylic acid for the treatment of colon-related disorders. In particular, a solid dosage form having a bilayer coating for effective delayed release for the treatment of colonic disorders is described.

### BACKGROUND

In the treatment of diseases or ailments of the colon or rectum, administration of the pharmacologically active agent to the affected site may be required. Orally administrable pharmaceutical compositions however have frequently been found ineffective in this respect as a result of the absorption or degradation of the pharmacologically active agent in the digestive tract before the colon or rectum is reached. Consequently, the delivery of pharmacologically active agents to the colon or rectum has conventionally been achieved by rectal administration, by the use of either suppositories or enemas. However, rectal administration generally is less convenient and less acceptable to a patient than oral administration. Further, said rectal administration is not suitable for treating the right side of the colon. In particular, suppositories are only effective for the rectum and enemas rarely reach beyond the left side of the colon.

### SUMMARY

The present invention comprises a delayed release oral composition delivery system for the treatment of colonic disorders and diseases such as ulcerative colitis and Crohn's disease. In accordance with one aspect of the invention, a delayed release oral pharmaceutical composition comprises an active core comprising a therapeutically effective amount of 5-amino salicylic acid (i.e., mesalamine); a primary coating composition disposed around the active core, wherein the primary coating composition comprises an enteric polymer; and a secondary coating composition disposed around the primary coating composition, wherein the secondary coating composition comprises ethyl cellulose and hydroxypropyl methylcellulose. The primary and secondary coating compositions are functional to facilitate a gradual and continuous release of the active core over time that is targeted for an upper portion of the gastro-intestinal tract or upstream of the colon.

According to another aspect of the invention, the enteric polymer comprises polyvinyl acetate phthalate and is soluble at about pH 5.0 and above. The secondary coating composition comprises from about a 15:85 to about a 60:40 ratio mixture of ethyl cellulose and hydroxypropyl methylcellulose and is permeable at a pH below 7.0 due to the presence of hydroxypropyl methylcellulose. Therefore, the secondary coating composition is permeable in gastric juices and facilitates the gradual, metered release of the oral composition, and, in particular embodiments, the ratio of ethyl cellulose to hydroxypropyl methylcellulose can be any one of the following: 15:85, 25:75, 50:50, and 60:40.

According to still another aspect of the invention, the thickness of the primary coating composition as applied around the active core can be between about 98 microns and about 211 microns. The thickness of the secondary coating composition as applied around the primary coating can be between about 125 microns and about 211 microns. The total thickness of the primary and secondary coating compositions can be between about 269 microns and about 443 microns.

In addition to the active pharmaceutical ingredient in the active core, a combination of pharmaceutically acceptable adjuvants, disintegrants, binders, glidants, lubricants and/or diluents can be included to produce the oral composition such as in tablet form.

According to yet another aspect of the invention, a process for preparing the delayed release oral composition comprises the steps of:

(a) screening lactose, povidone, and intra granular sodium starch glycolate through a mill;
(b) adding step (a) to 5-amino salicylic acid;
(c) dry mixing step (b);
(d) granulating step (c) with purified water and drying at 70° C. to moisture of about 0.1 to 1.0%;
(e) milling step (d) with extragranular sodium starch glycolate and magnesium stearate and mixing in a blender;
(f) compressing using a tablet press;
(g) applying a coat of polyvinyl acetate phthalate/simethicone dispersion to step (f);
(h) applying a coat of ethyl cellulose/HPMC dispersion to step (g); and
(i) optionally imprinting using standard imprinting ink on step (h).

These and other aspects, as well as a further understanding of the invention will be had from the following detail description and non-limiting examples. These aspects are indicative, however, of but a few of the various ways in which the principles of the invention may be employed and the subject invention is intended to include all such aspects and their equivalents. Other advantages and novel features of the invention may become apparent from the following detailed description of the invention.

### DETAILED DESCRIPTION OF THE INVENTION

The present invention relates to a delayed-release oral pharmaceutical composition for treatment of colonic disorders. The oral pharmaceutical composition comprises three components:

(1) an active core comprising 5-amino-salicylic acid (5-ASA);

(2) a primary coating composition disposed around the active core; and

(3) a secondary coating composition disposed around the primary coating.

The primary and secondary coatings can also be described as a bilayer coating disposed around the active core. Each of the three components can be described as follows.

### Active Core

The (coated) active core contains a concentration of about 65% w/w of 5-ASA (active ingredient) or in the alternative, about 50% w/w to about 80% w/w of 5-ASA, which is also referred to as mesalamine. In its uncoated form, the active core has a concentration of about 65% w/w to about 95% w/w of 5-ASA. In addition to the active ingredient, the core can also include one or more pharmaceutically accepted adjuvants. Pharmaceutically acceptable adjuvants include, but are not limited to, disintegrants, diluents, lubricants, glidants, and blends thereof. The disintegrants may be contained in the granules of the subject oral composition (intragranular) or may be separately mixed with the granules (extragranular). The extragranular disintegrants are preferably rapidly acting disintegrants, for example sodium starch glycolate, croscarmellose sodium, croscarmellose calcium or crospovidone. Diluents, such as lactose, starch, other cellulose derivatives, calcium carbonate, calcium phosphate dibasic (anhydrous, dihydrate), calcium-phosphate tribasic, magnesium carbonate, maltose, sorbitol, pregelatinized starch, sucrose, and compressible sugar, increase the bulk of a solid pharmaceutical composition, and may make a pharmaceutical dosage form containing the composition easier for the patient and/or a care giver to handle.

Glidants and lubricants may be added to a formulation to improve the flowability of a powder blend, reduce powder adhesion to equipment, and to improve the consistency of dosage weight. Adjuvants that may function as glidants include colloidal silicon dioxide, magnesium silicate, powdered cellulose, starch, talc, and tri-basic calcium phosphate, and mixtures thereof. Lubricants include magnesium stearate, calcium stearate, glyceryl monostearate, glyceryl palmitostearate, glyceryl behenate, hydrogented castor oil, hydrogenated vegetable oil, mineral oil, polyethylene glycol, sodium benzoate, sodium lauryl sulfate, sodium stearyl fumarate, stearic acid, talc, zinc stearate, and mixtures thereof. A binder such as starch, natural gums, such as gum acacia, gelatin, tragacanth, glucose, molasses, polyvinyl pyrrolidine, copovidone, sodium CMC, ethylcellulose, guar gum, poly dextrose, polymethacrylates, sodium alginate, sucrose, celluloses and derivatives thereof, povidone, and other such binders known to those of skill in the art can also be included in the active core.

The active ingredient is preferably combined intragranularly with about 5.0% w/w to about 22.0% w/w of lactose (diluent), about 0.1% w/w to about 4.0% w/w of sodium starch glycolate (disintegrant), and about 1.0% w/w to about 5.0% w/w of povidone (binder) and purified water (granulation fluid). In a particular embodiment, the amount of lactose may be about 13.5% w/w; the amount of sodium starch glycolate may be about 1.5% w/w; and the amount of povidone may be about 2.4% w/w.

An extragranular disintegrant and lubricant including sodium starch glycolate and magnesium stearate, respectively, can also be contained in the active core. The extragranular disintegrant can have a concentration from about 0.1% w/w to about 4.0% w/w and the extragranular lubricant may have a concentration from about 0.25% w/w to about 2.0% w/w of the total weight of the oral pharmaceutical composition. In a particular embodiment, both the extragranular disintegrant and lubricant can have the same concentration of about 0.7% w/w, respectively.

### Primary Coating Composition

The primary coating composition includes an enteric polymer that is soluble at a pH of at least 5.0. Examples include but are not limited to polyvinyl acetate phthalate which is commercially available under the name Sureteric®, cellulose acetate trimellitate, hydroxypropyl methylcellulose phthalate, hydroxypropylmethylcellulose acetate succinate, methacrylic acid copolymer (Type C) (e.g., Eudragit® L 100-55), methacrylic acid copolymer dispersion (e.g., Eudragit® L 30D-55), cellulose acetate phthalate, methacrylic acid copolymer (Type B), Eudragit® FS 30 D (an aqueous based acrylic polymeric dispersion consisting of methacrylic acid, methyl acrylate, and methyl methacrylate) and shellac.

The enteric polymer comprises about 2% w/w to about 20% w/w of the total oral pharmaceutical composition, and in a particular embodiment, about 8.3% w/w of the total oral pharmaceutical composition. An antifoam agent such as a 30% simethicone emulsion or dimethicone can also be included in the primary coating composition in a concentration from about 0.01% w/w to about 0.20% w/w, and in a particular embodiment, in a concentration of about 0.03% w/w.

Furthermore, the primary coating composition can be disposed around the active core at a thickness of about 98 to about 211 microns. The primary coating composition is preferably soluble at pH 5.0 and above in order to protect from and/or mitigate release of the drug's active core in the patient's stomach. In addition, the gastric resistant enteric polymer employed herein can eliminate large variations in initial release time in the gastrointestinal tract, especially in the stomach.

### Secondary Coating Composition

The secondary coating composition is disposed around the primary coating at a thickness of about 125 to about 211 microns, whereby a total coating thickness of both the primary and secondary coatings can be about 269 to about 443 microns. Included in the secondary coating composition is a polymer such as a combination of a highly permeable polymer and a sparingly permeable polymer, wherein the highly permeable polymer functions as a channelling agent (i.e., provides enhanced permeability to gastric or intestinal fluids relative to said sparingly permeable polymer). An example of such a combination includes an ethyl cellulose-based polymer and hydroxypropyl methylcellulose (HPMC). One example of the ethyl cellulose-based polymer is marketed under the name Surelease® and HPMC is commercially available under the name Opadry® II. Another exemplary combination can be found under the commercial names Eudragit® RL 30 D and Eudragit® RS 30 D—chemically referred to as an ammonio methacrylate copolymer dispersion of Type A and Type B, respectively.

The secondary coating composition is pH-independent and the polymer included herein provides a gradual release of the active core upstream of the colon or in an upper portion of the gastrointestinal tract. The polymer can include a mixture of an ethyl cellulose-based polymer and HPMC at a ratio ranging from about 15:85 to about 60:40, respectively; however, it should be appreciated that other ratio mixtures of the ethyl cellulose-based polymer to HPMC are feasible as well. In a particular embodiment, the other mixture ratios can include but are not limited to 15:85, 25:75, 50:50, and 60:40.

In addition to providing tablet elegance, the combination of Surelease® and Opadry®, for example, provide the preferred release rate with Opadry® functioning as a channelling agent in the ethyl cellulose film. Flavoring agents and flavor enhancers can make the dosage form more palatable to the patient. Thus, they can be included in the composition such as in the primary and/or secondary coatings. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

### Exemplary Delayed Release Oral Composition

| | | | |
|---|---|---|---|
| | | Best | |
| | | Scenario | Range |
| Ingredients | Purpose | (% w/w) | (% w/w) |

Intragranular (Tablet Core)

| | | | |
|---|---|---|---|
| Mesalamine | Active | 65.0 | 50.0-80.0 |
| | Ingredient | | |
| Lactose | Diluent | 13.5 | 5.0-22.0 |
| Sodium Starch Glycolate | Disintegrant | 1.5 | 0.1-4.0 |
| Povidone | Binder | 2.4 | 1.0-5.0 |
| Water Purified | Granulation Fluid | * | * |

Extragranular (Tablet Core)

| | | | |
|---|---|---|---|
| Sodium Starch Glycolate | Disintegrant | 0.7 | 0.1-4.0 |
| Magnesium Stearate | Lubricant | 0.7 | 0.25-2.0 |
| Theoretical Core Tablet | | 515.0 | |
| Weight | | | |

Primary Coat Composition

| | | | |
|---|---|---|---|
| Sureteric, YAE-6-18107 | Enteric Polymer | 8.3 | 2.0-20.0 |
| Simethicone Emulsion 30% | Antifoam | 0.03 | 0.01-0.2 |
| Water Purified | Vehicle | * | * |

Secondary Coat Composition

| | | | |
|---|---|---|---|
| Surelease, E-7-19010 Clear | pH independent | 3.9 | 1.0-12.0 |
| | control release | | |
| | polymer | | |
| Opadry II, Y-22-14001 (Pink) | Film Coating | 3.9 | 1.0-12.0 |
| Coating Material | Polymer/ | | |
| | Channeling Agent | | |
| Water Purified | Vehicle | * | * |

Imprinting

| | | | |
|---|---|---|---|
| Opacode S-1-8090 | Imprinting Ink | ** | ** |
| Theoretical Coated and | | 615.0 | |
| Imprinted Tablet Weight (mg) | | | |

* Removed during the manufacturing process
** Negligible amount

### Manufacturing Process

According to one aspect of the invention, the delayed release oral pharmaceutical composition can be manufactured into a tablet form as follows:
(1) screen lactose, povidone, and intra granular sodium starch glycolate through a mill;
(2) add mesalamine to step (1);
(3) dry mix step (2) in a granulator;
(4) granulate step (3) with purified water and dry at 70° C. to moisture of about 0.1 to 1.0%;
(5) mill step (4) with extragranular sodium starch glycolate and magnesium stearate and mix in a blender;
(6) compress using tablet press;
(7) apply coat of polyvinyl acetate phthalate (e.g., Sureteric®)/Simethicone dispersion to step (6);
(8) apply coat of ethyl cellulose/HPMC (e.g., Surelease®/Opadry II®) dispersion to step (7); and
(9) imprint using standard imprinting ink on step (8).

Delivery of the tablet is targeted in the upper gastrointestinal tract (e.g., upstream of the colon). The precise location may vary depending on the condition of a patient such as their current health, weight, size, disease state, and/or fasted conditions. However, due to the unique pH characteristics of the primary and secondary coatings, the active core is released continuously by diffusion over time. The secondary coating composition is pH-independent and begins releasing in gastric and intestinal juice. The primary coating is pH-dependent and is soluble at a pH above 5.0 and dissolves to allow 100% release of the active ingredient between a pH of 5.8 to 6.8. Conventional delivery systems for 5-ASA compositions used in the treatment of colonic disorders are insoluble in gastric and intestinal juices at a pH just below 7.0 and are rapidly soluble in environments where the pH is at least 7.0 such as in colonic intestinal juices.

According to another aspect of the invention, the primary and secondary coating compositions for disposition over the active core of the delayed release oral pharmaceutical composition can be manufactured and applied to the active core as follows:

For the Primary Coating:
(1) dispense 23.612 kg of polyvinyl acetate phthalate (Sureteric®).
(2) dispense 78.20 g of simethicone emulsion 30% USP.
(3) dispense 134.244 kg of purified water USP.

For the Secondary Coating:
(4) dispense 44.620 kg of ethyl cellulose (e.g., Surelease®).
(5) dispense 11.156 kg of HPMC (e.g., Opadry® II).
(6) dispense 92.970 kg of purified water USP.

The Primary Coating Manufacturing Process:
(7) To a prepared tank, add the purified water (134.244 kg).
(8) Add simethicone emulsion 30% USP (78.2 g) to the step (7) tank.
(9) Disperse step (8) for a minimum of 5 minutes.
(10) Add 23.612 kg of polyvinyl acetate phthalate to the tank of step (9).
(11) Disperse step (10) for a minimum of 5 minutes.
(12) Mix step (11) for a minimum of 90 minutes.
(13) While mixing step (12), screen step (12) through a 60 mesh stainless steel screen and transfer into a tank.
(14) Mix step (13) for a minimum of 15 minutes.
(15) If not proceeding directly to step (16), mix step (14) for a minimum of 15 minutes just prior to use.
(16) Film coat the uncoated tablets in a coating pan using the step (14) or (15) prepared coating dispersion.

For the Secondary Coating Manufacturing Process:
(17) To a prepared tank, add the purified water (92.970 kg).
(18) Add HPMC (11.156 kg) to the step (17) tank.
(19) Disperse step (18) for a minimum of 60 minutes.
(20) Add ethyl cellulose (44.620 kg) to the tank of step (19).
(21) Disperse step (20) for a minimum of 60 minutes.
(22) If not proceeding directly to step (23), mix step (21) for a minimum of 15 minutes just prior to use.
(23) Film coat the step (16) primary coated tablets in a coating pan using the step (21) or (22) prepared coating dispersion.

### Dissolution Data Based on Secondary Coating Composition Ratio

In a first series of tests, four mesalamine delayed release 400 mg tablets were dissolved in a pH 6.8 buffer solution to demonstrate the release of the active core over an 8-hour period based on the ratio of ethyl cellulose to HPMC (Surelease® to Opadry®) in the secondary coating composition. Each tablet had an active core composed of the following materials:

| | |
|---|---|
| 5-Aminosalicylic Acid, USP Coarse | 400.0 mg |
| Lactose NF (Regular) | 83.0 mg |
| Sodium Starch Glycolate NF | 13.0 mg |
| Povidone, USP | 15.0 mg |
| Magnesium Stearate NF | 4.0 mg |

As shown in Table 1, a percentage amount released of the active core is further delayed as the amount of ethyl cellulose increases and the amount of the HPMC decreases.

TABLE 1
% of Active Core Released in Hours

| Ratios | | | | | | | | | |
|---|---|---|---|---|---|---|---|---|---|
| (Surelease:Opadry) | 0 | 1 | 2 | 3 | 4 | 5 | 6 | 7 | 8 |
| 15:85 | 0 | 4 | 68 | 101 | 102 | 102 | | | |
| 25:75 | 0 | 2 | 73 | 101 | 102 | 101 | | | |
| 50:50 | 0 | 0 | 0 | 0 | 10 | 80 | 100 | | |
| 60:40 | 0 | 0 | 0 | 0 | 0 | 0 | 11 | 53 | 99 |

### Example

In another series of tests conducted in pH 6.5 and pH 6.0 buffer solutions, 12 and 6 mesalamine delayed release tablets, respectively, were dissolved—each with the same core tablet and coating compositions. The manufacture and coating processes employed to produce the mesalamine delayed release tablets is described accordingly below.

The composition of each tablet not including the tablet coatings (hereinafter referred to “core tablet”) was as follows in Table 2:

TABLE 2

| Total Tablet Dosage | | Material | | |
|---|---|---|---|---|
| (Amount per Tablet) | | (Intragranular) | Quantity Dispensed | |
| 400.0 | mg | 5-Aminosalicylic Acid, USP | 92.010 | kg |
| | | Coarse | | |
| 83.0 | mg | Lactose NF (Regular) | 19.090 | kg |
| 9.0 | mg | Sodium Starch Glycolate NF | 2.070 | kg |
| 15.0 | mg | Povidone, USP | 3.450 | kg |

| | | | |
|---|---|---|---|
| QS* | Water, Purified USP | 64.950 | kg |

*Processing component which does not appear in the final product.

To produce the core tablets, the preceding intragranular materials were subjected to a compounding process followed by a final blending with the extragranular materials listed below in Table 3:

TABLE 3

| Total Tablet Dosage | Material | |
|---|---|---|
| (Amount per Tablet) | (Extragranular) | Quantity Dispensed |
| 4.0 mg | Sodium Starch Glycolate NF | 920 g |
| 4.0 mg | Magnesium Stearate, NF | 920 g |

After the final blending was completed, the blended materials underwent a compression process to form the tablets alone. Thereafter, the formed tablets were divided into Sub Lot A and Sub Lot B and were ready to be coated initially with a primary coating composition. The primary coating composition was dispensed as stated in Table 4 below:

TABLE 4

| Dosage | | | |
|---|---|---|---|
| (Amount per Tablet) | Material | Quantity Dispensed | |
| 51.33 mg | Sureteric | 11.806 | kg |
| 0.17 mg | 30% Simethicone Emulsion, | 39.10 | g |
| | USP | | |
| QS* | Water, Purified USP | 67.122 | kg |

*Processing component which does not appear in the final product.

Sub Lot A was coated with the primary coating composition using the following process:
(1) 67.122 kg of the purified water was added to a tank.
(2) 39.10 g of the 30% Simethicone Emulsion USP was added to the step (1) tank.
(3) Step (2) was dispersed for 5 minutes.
(4) With a scoop, 11.806 kg of Sureteric® was added to the tank of step (3).
(5) Step (4) was dispersed for 5 minutes.
(6) Step (5) was mixed for 90 minutes.
(7) While step (6) was mixed, step (6) was screened through a 60 mesh stainless steel screen into a tank equipped with an agitator.
(8) Step (7) continued to be mixed for 15 minutes.
(9) Film coated the Sub Lot A core tablets using the prepared coating dispersion of step (8).

Sub Lot B of the above manufactured tablets was coated with the primary coating composition using the following process:
(10) 67.122 kg of the purified water was added to a tank.
(11) 39.10 g of the 30% Simethicone Emulsion USP was added to the step (10) tank.
(12) Step (11) was dispersed for 5 minutes.
(13) With a scoop, 11.806 kg of Sureteric® was added to the step (12) tank.
(14) Step (13) was dispersed for 5 minutes.
(15) Step (14) was mixed for 90 minutes.
(16) While step (15) was mixed, step (15) was screened through a 60 mesh stainless steel screen into a tank equipped with an agitator.
(17) Step (16) continued to be mixed for 15 minutes.
(18) Film coated the Sub Lot B core tablets using the prepared coating dispersion of step (17).

The secondary coating composition used to coat the primary coated Sub Lot A and Sub Lot B tablets was dispensed according to Table 5:

TABLE 5

| Dosage | | |
|---|---|---|
| (Amount per Tablet) | Material | Quantity Dispensed |
| 24.25 mg | Surelease ® | 22.310 kg |
| 24.25 mg | Opadry ® | 5.578 kg |
| QS* | Water, Purified USP | 46.500 kg |

*Processing component which does not appear in the final product.

To coat the Sub Lot A primary coated tablets with the secondary coating composition, the following process was employed:
(19) 46.500 kg of purified water was added to a tank.
(20) 5.578 kg of Opadry® was added to the step (19) tank.
(21) Dispersed step (20) for 60 minutes.
(22) 22.310 kg of Surelease® was added to the tank of step (21).
(23) Dispersed step (22) for 60 minutes.
(24) Film coated the step (9) primary coated Sub Lot A tablets using the prepared secondary coating dispersion from step (23).

To coat the Sub Lot B primary coated tablets with the secondary coating composition, the following process was employed:
(25) To a tank, 46.485 kg of purified water was added.
(26) 5.578 kg of Opadry® was added to the step (25) tank.
(27) Dispersed step (26) for 60 minutes.
(28) 22.310 kg of Surelease® was added to the tank of step (27).
(29) Dispersed step (28) for 60 minutes.
(30) Film coated the step (18) primary coated Sub Lot B tablets using the prepared secondary coating dispersion from step (29).

Following therefrom, 12 mesalamine delayed release tablets, as produced via the above processes, were dissolved in a pH 6.5 buffer solution in order to observe the rate of dissolution. The results are depicted in Table 6 and illustrate the gradual release of the delayed release oral composition described hereinabove at a pH below 7.0.

TABLE 6
% of Active Core Released in Minutes (pH 6.5)

| | 360 | 420 | 480 | 540 | 600 | 660 | 720 | 780 | 840 | 900 | 960 | 1020 |
|---|---|---|---|---|---|---|---|---|---|---|---|---|
| 1 | 0 | 2 | 24 | 56 | 79 | 87 | 94 | 99 | 101 | 103 | 106 | 109 |
| 2 | 0 | 0 | 0 | 2 | 25 | 59 | 78 | 89 | 95 | 101 | 104 | 111 |
| 3 | 0 | 0 | 4 | 15 | 44 | 75 | 95 | 105 | 107 | 108 | 110 | 115 |
| 4 | 0 | 0 | 0 | 0 | 1 | 0 | 39 | 69 | 84 | 92 | 101 | 107 |
| 5 | 0 | 0 | 1 | 17 | 53 | 87 | 107 | 112 | 112 | 113 | 114 | 117 |
| 6 | 0 | 0 | 0 | 1 | 45 | 87 | 94 | 102 | 105 | 109 | 112 | 111 |
| 7 | 0 | 0 | 2 | 9 | 46 | 74 | 77 | 103 | 111 | 113 | | |
| 8 | 0 | 0 | 2 | 15 | 48 | 80 | 90 | 95 | 103 | 113 | | |
| 9 | 6 | 36 | 73 | 99 | 99 | 104 | 104 | 106 | 106 | 110 | | |
| 10 | 0 | 0 | 1 | 2 | 6 | 63 | 89 | 109 | 113 | 120 | | |
| 11 | 0 | 0 | 4 | 43 | 68 | 84 | 90 | 93 | 98 | 107 | | |
| 12 | 0 | 0 | 2 | 31 | 74 | 87 | 101 | 105 | 108 | 110 | | |

Similarly, 6 mesalamine delayed release tablets as produced via the above processes were dissolved in a pH 6.0 buffer solution in order to observe the rate of dissolution. The dissolution results are indicated in Table 7. Unlike conventional pharmaceutical compositions that treat colonic diseases such as ulcerative colitis, the delayed release oral composition as discussed herein gradually releases at a pH well below 7.0 as indicated in Table 7. This data further demonstrates that the subject oral composition is gradually released upstream of the colon in the gastric juices of the upper gastro-intestinal tract.

TABLE 7
% of Active Core Released in Hours (pH 6.0)

| | 1-3 | 4 | 5 | 6 | 7 | 8 | 9 | 10 | 11 | 12 | 13 | 14 |
|---|---|---|---|---|---|---|---|---|---|---|---|---|
| 1 | 0 | 0 | 0 | 0 | 0 | 0 | 16 | 84 | 91 | 94 | 94 | 94 |
| 2 | 0 | 0 | 0 | 0 | 0 | 0 | 0 | 4 | 15 | 90 | 91 | 91 |
| 3 | 0 | 0 | 0 | 0 | 0 | 0 | 46 | 88 | 90 | 93 | 94 | 93 |
| 4 | 0 | 0 | 0 | 0 | 0 | 0 | 36 | 89 | 92 | 92 | 92 | 92 |
| 5 | 0 | 0 | 0 | 0 | 0 | 0 | 43 | 90 | 92 | 92 | 94 | 92 |
| 6 | 0 | 0 | 0 | 0 | 0 | 0 | 86 | 94 | 94 | 94 | 93 | 93 |

While various embodiments in accordance with the present invention have been shown and described, it is understood the invention is not limited thereto, and is susceptible to numerous changes and modifications as known to those skilled in the art. Therefore, this invention is not limited to the details shown and described herein, and includes all such changes and modifications as encompassed by the scope of the appended claims.

What is claimed is:

1. A delayed release oral pharmaceutical composition comprising:
an active core comprising a therapeutically effective amount of 5-amino salicylic acid;
a primary coating composition disposed around the active core, wherein the primary coating composition comprises an enteric polymer that is soluble at a pH of at least 5.0; and
a secondary coating composition disposed around the primary coating composition, wherein the secondary coating composition comprises a highly permeable polymer and a sparingly permeable polymer, wherein the highly permeable polymer is adapted to provide enhanced permeability to gastric or intestinal fluids relative to the sparingly permeable polymer.

2. The composition of claim 1, wherein the enteric polymer is selected from at least one of the following: polyvinyl acetate phthalate, cellulose acetate trimellitate, hydroxypropyl methylcellulose phthalate, hydroxypropylmethylcellulose acetate succinate, methacrylic acid copolymer (Type C), methacrylic acid copolymer dispersion, cellulose acetate phthalate, methacrylic acid copolymer (Type B), and shellac.

3. The composition of claim 1, wherein the secondary coating composition comprises at least one of the following ratio mixtures of highly permeable polymer to sparingly permeable polymer: 15:85, 25:75, 50:50, and 60:40.

4. The composition of claim 1, wherein the secondary coating composition comprises an ethyl cellulose-based polymer and hydroxypropyl methylcellulose.

5. The composition of claim 4, wherein the secondary coating composition comprises about a 50:50 mixture of the ethyl cellulose-based polymer and hydroxypropyl methylcellulose, wherein the ethyl cellulose is about 1.0% w/w to about 12% w/w and the hydroxypropyl methylcellulose is about 1.0% w/w to about 12% w/w.

6. The composition of claim 4, wherein the secondary coating composition comprises any one of the following ratio mixtures of the ethyl cellulose-based polymer to hydroxypropyl methylcellulose: 15:85, 25:75, 50:50, and 60:40 ratio mixtures.

7. The composition of claim 1, wherein the secondary coating composition comprises ammonio methacrylate copolymer dispersion of Type A and Type B.

8. The composition of claim 1, wherein the primary coating composition is disposed around the active core at a thickness between about 98 microns and about 211 microns.

9. The composition of claim 1, wherein the secondary coating composition is disposed around the primary coating composition at a thickness between about 125 microns and about 211 microns.

10. The composition of claim 1 has a combined primary and secondary coating composition thickness between about 269 microns and about 443 microns.

11. The composition of claim 1, wherein the enteric polymer is polyvinyl acetate phthalate.

12. The composition of claim 1, wherein the enteric polymer is about 8.3% w/w of the delayed release oral pharmaceutical composition.

13. The composition of claim 1, wherein the enteric polymer is between about 2% w/w to about 20% w/w of the delayed release oral pharmaceutical composition.

14. The composition of claim 1, wherein the active core comprises about 50% w/w to about 80% w/w of 5-amino salicylic acid.

15. The composition of claim 1, wherein the active core further comprises intra granular lactose, sodium starch glycolate, and povidone and extra granular magnesium stearate and sodium starch glycolate.

16. The composition of claim 1 is in a tablet form.

17. The composition of claim 1, wherein the primary coating composition further comprises about 0.01% w/w to about 0.20% w/w of a 30% simethicone emulsion.

18. A method for treating ulcerative colitis and/or Crohn's disease in a patient in need thereof comprising administering to said patient a composition according to claim 1.

19. The method of claim 18, wherein an active core is targeted for and meter released over time beginning upstream of a patient's colon.

20. A delayed release oral pharmaceutical composition comprising:
an active core comprising a pharmaceutically acceptable amount of 5-amino salicylic acid;
a primary coating composition disposed around the active core, wherein the primary coating composition comprises polyvinyl acetate phthalate; and
a secondary coating composition disposed around the primary coating composition, wherein the secondary coating composition comprises about a 50:50 ratio of ethyl cellulose and hydroxypropyl methylcellulose.

21. A process for preparing a delayed release oral pharmaceutical composition, the process comprising:
(a) screening lactose, povidone, and intra granular sodium starch glycolate and lactose through a mill;
(b) adding 5-amino salicylic acid to step (a);
(c) dry mixing step (b);
(d) granulating step (c) with purified water and dry at 70° C. to moisture of about 0.1% to 1.0%;
(e) milling step (d) with extragranular sodium starch glycolate and magnesium stearate and mix in a blender;
(f) compressing using tablet press;
(g) applying coat of polyvinyl acetate phthalate/simethicone dispersion to step (f); and
(h) applying coat of ethyl cellulose/HPMC dispersion to step (g).

22. The process of claim 21, wherein the polyvinyl acetate phthalate/simethicone dispersion is applied at a thickness between about 98 microns and about 211 microns and wherein the ethyl cellulose/HPMC dispersion is applied at a thickness between about 125 microns and about 211 microns.

23. A product produced in accordance with the process of claim 21 for treatment of colonic disorders.
